(19) United States
(12) Patent Application Publication
(10) Pub. No.: US 2012/0302577 A1
(43) Pub. Date: Nov. 29, 2012

(54) TREATMENT OF GIST WITH MASITINIB

(75) Inventors: Alain Moussy, Paris (FR); Jean-Pierre Kinet, Aix En Provence (FR)

(73) Assignee: AB SCIENCE, PARIS (FR)

(21) Appl. No.: 13/575,722

(22) PCT Filed: Jan. 28, 2011
(86) PCT No.: PCT/EP11/51187

§ 371 Date: Jul. 27, 2012

Related U.S. Application Data
(60) Provisional application No. 61/298,953, filed on Jan. 28, 2010.

(51) Int. Cl. A61K 31/496; A61K 31/506; C07D 417/14; A61P 35/00; A61P 35/04

(52) U.S. Cl. 514/252.18; 514/253.1; 544/364

(57) ABSTRACT

The present invention relates to the use of masitinib or a pharmaceutically acceptable salt thereof, and in particular of masitinib mesylate, for the preparation of a medicament for the treatment of GIST, to the use of this therapy for the treatment of GIST, and a method of treating mammals, including humans, suffering from GIST by administering to said mammal in need of such treatment an effective dose of masitinib, and in particular masitinib mesylate.

### FIELD OF THE INVENTION

The invention relates to a method for the treatment of GIST.

The method of the invention comprises the administration of masitinib, or pharmaceutically acceptable salt thereof.

### BACKGROUND OF THE INVENTION

### Definition and Terminology

Gastrointestinal stromal tumours (GIST) are usually defined as specific, generally Kit positive and Kit and PDGFRA mutation-driven mesenchymal tumours of the gastrointestinal (GI) tract with a set of characteristic histological features including spindle cell, epithelioid and rarely pleomorphic morphology.

### Description of GIST

GIST are rare sarcoma tumours affecting the digestive tract and nearby structures within the abdomen. They arise from interstitial cells of Cajal or their precursors. GISTs are usually attached to the outside of the involved organ, growing outward.

Primary GIST may occur anywhere along the gastrointestinal tract from the oesophagus to the anus. The most frequent site is the stomach (˜55%) followed by the duodenum and small intestine (˜30%), oesophagus (˜5%), rectum (˜5%), colon (˜2%) and rare other locations.

Occasionally, primary GIST may develop in the supporting membranes of the abdominal organs (peritoneum, mesentery, omentum), the liver, the pancreas, the ovaries, the uterus and the prostate. Because these primary GISTs do not arise directly from the GI tract, they are sometimes called extragastrointestinal stromal tumour. GISTs not encased in the peritoneal membranes are called retroperitoneal. The most common sites for metastasis are the liver and the abdominal membranes (peritoneum, mesentery, omentum). GISTs rarely spread to lymph nodes, but they may occasionally affect local abdominal lymph nodes. Unusual sites of metastasis include lung and bones as well as pelvic sites such as the ovaries. Extremely rare sites include breast and muscle tissue.

### Incidence of GIST

Of all adult cancers, sarcomas represent about 1%; GIST is one of the most common of about 50 types of sarcomas. Age-adjusted incidence could be estimated about 6 to 15 per million.

### Risk Factors for GIST

No environmental or behavioural risk factors contributing to GIST have been identified. They appear to be no relationship between diet and lifestyle and the incidence of GIST.

### Affected Population for GIST

GISTs most commonly affect older people, usually over 50. They have a broad distribution with no gender or racial predilection.

### Symptoms of GIST

The most common presentation of GIST is gastrointestinal bleeding that may be acute or chronic insidious bleeding leading to anaemia.

Many patients with smaller tumours don't have symptoms. Larger tumours may cause symptoms that are generally related to the increased mass being accommodated in the abdominal cavity (digestive discomfort, sensation of abdominal fullness or abdominal pain); such symptoms would not necessarily be different from those of other types of tumours.

Sometimes larger tumours may be detectable by palpation. Some patients may experience vomiting or diarrhoea; bowel obstruction may occur. In case GIST perforate the stomach or gut lining and bleed into the GI tract, resulting in black or tarry stools and occasionally of vomiting of blood. Anaemia may result from chronic bleeding, leading to fatigue.

Though such symptoms are possible, most of them are rather indistinct and merely related to the additional mass. Therefore, many GISTs are found incidentally.

### Diagnosis of GIST

A final diagnosis can only be made immunohistochemically. GIST became a clear diagnosis only in 1998, when it was found that nearly all GIST cells express Kit and that many GIST show mutations in the Kit gene [1, 2]. About 70% of GIST are composed of spindle cells, while about 20% are composed of epithelioid cells and the other 10% show mixed cells of both spindle and epithelioid types. GIST can also contain activating mutations of the PDGFRA gene [3].

Kit-negative GIST are uncommon but about 5% do not stain for Kit. Several markers have been identified to help diagnose Kit-negative GIST (PKC beta and/or DOG1).

The pathologist can estimate proliferation; the higher the proliferation, the faster the tumour is growing and the more aggressively it can be expected to grow (if not resected) or to recur or metastasize (if removed).

Major negative factors include large size (>5 cm), high mitotic index and grossly positive resection margins. Other factors with poor prognosis include tumour rupture, high cellularity, tumour necrosis, presence of metastases or invasion and certain types of Kit mutations.

Factors that are correlated with an improved prognosis include gastric location, diameter less than 2 cm, low mitotic index and absence of tumour spillage with complete gross resection.

### Kit and PDGFRA Mutations in GIST

Mutations in the Kit gene that are relevant for GIST are found in exon 9, 11, 13 and 17, with mutation in exon 11 being the most frequent. Mutation in Kit can be found in about 80% of GIST.

About 8% of GISTs have a Kit WT but show mutations in PDGFRA. The PDGFRA gene is very similar to the Kit gene, and PDGFRA mutations have been found in exons corresponding to those of Kit.

Mutations of Kit and of PDGFRA are mutually exclusive in primary, untreated GIST.

### Secondary Mutations in Drug Resistance in GIST Treatment

Newly acquired secondary mutations have been shown to confer drug resistance to imatinib. They often appear in new metastases of tumours being treated with imatinib and in sections of otherwise responding tumours that start to grow.

Long-term success is limited by the development of imatinib resistance via secondary mutation or clonal selection.

### Management/Treatments of GIST

Excision of the tumour, when feasible is the treatment of choice. Patients whose tumours are unresectable or who have metastatic disease are treated with Kit/PDGFRA tyrosine kinase inhibitors such as imatinib. This oral treatment is generally well tolerated and the majority of patients achieve complete or partial remission.

Recent data suggest that the response of GIST patients to tyrosine kinase inhibitor varies by the specific mutation displayed by their tumours.

There is a stronger and longer-duration response to imatinib for patients with mutation in exon 11 than for those with mutations in exon 9 or for patients with GIST negative for Kit expression.

Response to sunitinib in patients who had grown resistant to imatinib was better for patients with exon 9 mutation.

### Remaining Problems in GIST Treatments

Imatinib represented a revolution for the treatment of patients with GIST, by improving the outcome of patients with advanced GIST from pre-imatinib 2-year survival rate of 25% to about 70% after its introduction. However, patients eventually progress and the majority of patients will die from their disease, despite an increase of imatinib daily dose from 400 mg to 800 mg or a switch to second-line therapy (e.g. sunitinib). These progressions are considered as late progression, to be distinguished from early progression (occurring within 3 to 6 months of treatment, in patients who never have a response to treatment).

Late progression is defined as progressions occurring in patients who had a response or a progression-free survival (PFS) over 3 to 6 months after initiation of imatinib treatment. In this case, progression results from resistance mechanisms developing under imatinib pressure, mostly the occurrence of secondary Kit mutation (in 50-70% of patients showing late progression), predominantly in the region encoding the part of the receptor in the vicinity of the ATP-binding site or the kinase activating loop [4]. These mutations change Kit conformation, and the ability of imatinib to bind to and inhibit Kit is reduced. These secondary mutations appear with a higher frequency in Kit with exon 11 mutation than in Kit with exon 9 mutation.

Results from a phase 3 study (345 patients receiving the initial dose of 400 mg/day) show median PFS of 18 months (95% CI [16-21]) and a 2-year PFS rate of 46% (95% CI [36-47]). Median overall survival (OS) was 55 months (95% CI [47-62]) with 2-year survival of 72% (95% CI [67-77]) and 3-year survival of ˜61% [5].

A study exploring the relationship between imatinib plasma levels and long-term clinical outcomes has shown that imatinib trough plasma levels seem to be correlated with clinical benefits, including longer Time-to-Progression (TTP) for patients with higher trough plasma levels (>1,110 ng/mL) than for those with lower trough levels, and objective response [6]. In addition, sex was not a significant covariate but the average imatinib trough level in women seemed about 25% higher than in men [6]. This was consistent with the body weight difference between men and women, suggesting that the dosage of imatinib in mg/day provides lower plasma levels and therefore potentially lowered efficacy to patients with higher body weight. These lower doses of imatinib could favour the rapid emergence of imatinib-resistant clones, thus progression.

### Goal of the Invention

The invention aims to solve the technical problem of providing a new treatment for GIST, and particularly a treatment overcoming the remaining problems in GIST treatments of the prior art.

The invention further aims to solve the technical problem of providing a method for the treatment of a subject with a proliferative disease wherein a tyrosine kinase is affected, in particular where said subject has cells showing a mutant kit and/or mutant PDGFRA gene(s).

In particular, the invention aims to solve the technical problem of providing a method for the treatment of a subject with GIST.

The invention further aims to solve the technical problem of providing a method for the long-term treatment of a subject with GIST.

The invention further aims to solve the technical problem of providing a method for the treatment of a subject with non-pre-treated, inoperable, locally advanced or metastatic GIST.

The invention further aims to solve the technical problem of providing a method for the treatment of a subject having cells resistant to a treatment of a proliferative disease wherein tyrosine kinase is affected. In particular the invention aims to provide a treatment for a subject having cells resistant to a tyrosine kinase inhibitor, and in particular to imatinib.

The invention further aims to solve the technical problem of providing a new pharmaceutical use or method involving masitinib or a pharmaceutically acceptable salt thereof.

The invention aims to achieve all the above mentioned goals while meeting industrial, in particular pharmaceutical, needs notably in term of drug efficacy, safety and regulatory requirements.

### DESCRIPTION OF THE INVENTION

The present invention solves the above mentioned technical problems.

In particular, the invention relates to a method for the treatment of a subject with GIST, wherein said method comprises the administration of an effective amount of masitinib or a pharmaceutically acceptable salt thereof, in particular of masitinib mesylate, to said subject.

According to one embodiment, said treatment is for treating or preventing cancer cell metastasis.

Advantageously, said treatment comprises the oral administration of masitinib or a pharmaceutically acceptable salt thereof, in particular of masitinib mesylate, to a subject in need thereof.

A preferred effective amount of masitinib or a pharmaceutically acceptable salt thereof, in particular of masitinib mesylate, is a daily dose below 18 mg/kg of subject weight. A preferred effective amount of masitinib or a pharmaceutically acceptable salt thereof, in particular of masitinib mesylate, is a daily dose comprised between 1 mg/kg and 15 mg/kg of subject weight.

Advantageously, said treatment comprises the administration of masitinib or a pharmaceutically acceptable salt thereof, in particular of masitinib mesylate, to a subject in need thereof, at a dose from 3 mg/kg/day to 15 mg/kg/day, from 6 mg/kg/day to 12 mg/kg/day, in particular 7.5 mg/kg/day, 9 mg/kg/day or 10.5 mg/kg/day. Masitinib is given in mg/kg/day with respect to the subject (particularly patient) weight. Unexpectedly, these low doses of the compound of the invention provide good results with respect to the treatment of GIST in human patients.

It is meant by the compound of the invention: masitinib or a pharmaceutically acceptable salt thereof, in particular of masitinib mesylate.

The effective dose is preferably administered to a subject depending on their weight. This enables a more effective treatment.

Accordingly, the present invention relates to a method for the treatment of a subject with GIST, wherein said effective amount of masitinib or a pharmaceutically acceptable salt thereof, in particular of masitinib mesylate, is a daily dose depending on the patient weight.

Advantageously in the method of the invention the dose is administered in two intakes a day (“bis in die”, i.e. bid). Dosing in two intakes reduces gastrointestinal adverse reactions without affecting efficacy.

The invention further relates to a method for the treatment of a subject with GIST wherein said subject having cells showing a native kit and/or PDGFRA gene(s), comprising the administration of masitinib or a pharmaceutically acceptable salt thereof, in particular of masitinib mesylate.

The invention further relates to a method for the treatment of a subject with a proliferative disease wherein a tyrosine kinase is affected, said subject having cells showing a mutant kit and/or mutant PDGFRA gene(s), comprising the administration of masitinib or a pharmaceutically acceptable salt thereof, in particular of masitinib mesylate.

According to one embodiment, said mutation is a mutation conferring resistance to a tyrosine kinase, and in particular to imatinib drug treatment. Where reference is made to imatinib, it refers in especially to Imatinib mesylate, or Gleevec, or STI-571; as produced by Novartis, Basel, Switzerland.

The invention further relates to a method for the long-term treatment of a subject with GIST, wherein said method comprises the administration on a long-term of an effective amount of masitinib or a pharmaceutically acceptable salt thereof, in particular of masitinib mesylate, to said subject.

A long-term treatment is preferably a treatment over more than 12 months, and preferably more than 2 years. The treatment of the present invention extends the PFS (Progression-Free Survival).

The invention further relates to a method for the treatment of a subject with non-pre-treated, inoperable, locally advanced or metastatic GIST, wherein said method comprises the administration of an effective amount of masitinib or a pharmaceutically acceptable salt thereof, in particular of masitinib mesylate, to said subject. The invention relates in particular to a first-line therapy method wherein masitinib or a pharmaceutically acceptable salt thereof, in particular masitinib mesylate, is administrated to a patient in need thereof, and in particular to a patient, whose tumour is not treatable by surgery.

A preferred effective amount of masitinib or a pharmaceutically acceptable salt thereof, in particular of masitinib mesylate, is a daily dose from 6 mg/kg to 9 mg/kg of subject weight, preferably from 7 to 8 mg/kg of subject weight, and more preferably a dose of 7.5 mg/kg of subject weight. This dose is particularly preferred for a first-line therapy.

It has been discovered by the inventors that masitinib or a pharmaceutically acceptable salt thereof, in particular masitinib mesylate, inhibits the growth of cells resistant to another c-kit inhibitor, and in particular to imatinib-resistant cells.

Accordingly, the invention relates to a method wherein masitinib or a pharmaceutically acceptable salt thereof, in particular masitinib mesylate, is administrated in combination with another tyrosine kinase inhibitor, and in particular in combination with imatinib.

The invention also relates to a second-line therapy method, wherein masitinib or a pharmaceutically acceptable salt thereof, in particular masitinib mesylate, is administrated to a patient in need thereof, and in particular to a patient, whose tumour is resistant to another tyrosine kinase inhibitor, and in particular to imatinib.

A second-line therapy is a treatment that is given when initial treatment (first-line therapy) doesn't work, or stops working.

A preferred effective amount of masitinib or a pharmaceutically acceptable salt thereof, and in particular of masitinib mesylate, is a daily dose from 10.5 to 15 mg/kg of subject weight, preferably from 11.5 to 13.5 mg/kg of subject weight, and more preferably is a daily dose of 12.5 mg/kg of subject weight. This dose is particularly preferred for a second-line therapy.

Accordingly, the invention relates to a method of treatment of a patient in need thereof, wherein said method comprises a first-line treatment comprising the administration to said patient of a tyrosine kinase inhibitor, and in particular imatinib, and as a second line treatment the administration of masitinib or a pharmaceutically acceptable salt thereof, in particular masitinib mesylate,

The invention further relates to a method for the treatment of a subject having cells resistant to a treatment of a proliferative disease wherein tyrosine kinase is affected, said treatment comprises the administration of a tyrosine kinase inhibitor other than masitinib, said method comprising the steps of:
(i) identifying in a subject a cell resistance to a treatment by a tyrosine kinase inhibitor other than masitinib of a proliferative disease wherein tyrosine kinase is affected;
(ii) Administering masitinib or a pharmaceutically acceptable salt thereof, in particular masitinib mesylate, to said subject.
   The invention relates in particular to the treatment of a human being. Thus a subject to the treatment is in particular a human patient.

The invention further relates to masitinib or a pharmaceutically acceptable salt thereof, in particular masitinib mesylate as a medicament in a method as described above or below, without particular limitation, and including the examples and drawings.

The invention further relates to a pharmaceutical composition comprising masitinib or a pharmaceutically acceptable salt thereof, in particular masitinib mesylate, for a method as described above or below, without particular limitation, and including the examples and drawings.

According to a particular embodiment, the composition of the invention is an oral composition.

Advantageously, said composition is in the form of a plurality of unit doses for administering an effective daily dose of masitinib or a pharmaceutically acceptable salt thereof, in particular of masitinib mesylate to a human patient in need thereof, wherein said dose is administered in pharmaceutical composition comprising below 3000 mg, more particularly between 1 mg and 2500 mg, and more particularly the dose is from 25 mg to 2000 mg. A preferred dose is from 50 mg to 150 mg, more preferably from 80 to 120 mg, and even more preferably 100 mg, for a first-line treatment. A preferred dose is from 150 mg to 400 mg, more preferably from 180 to 300 mg, and even more preferably 200 mg, for a second-line treatment.

The doses described in the invention provide advantageously plasma levels high enough to inhibit Kit WT, Kit mutant forms involved in GIST and PDGFRA. In particular, weight-adjusted doses potentially provide all patients with the same masitinib plasma levels.

The invention further relates to masitinib or a pharmaceutically acceptable salt thereof, in particular masitinib mesylate, as an inhibitor of Kit and PDGFRA mutants for the treatment of a disease with kit and/or PDGFRA mutants. In particular masitinib or a pharmaceutically acceptable salt thereof, in particular masitinib mesylate, is an inhibitor of kit mutants with mutation in exon 9, and/or 11, and/or 13, and/or 17. In particular masitinib or a pharmaceutically acceptable salt thereof, in particular masitinib mesylate, is an inhibitor of PDGFRA mutants with mutation in exon 12, and/or 14, and/or 18.

As is known to the person skilled in the art, various forms of excipients can be used adapted to the mode of administration and some of them can promote the effectiveness of the active molecule, e.g. by promoting a release profile rendering this active molecule overall more effective for the treatment desired.

The pharmaceutical compositions of the invention are thus able to be administered in various forms, more specially for example in an injectable, pulverizable or ingestible form, for example via the intramuscular, intravenous, subcutaneous, intradermal, oral, topical, rectal, vaginal, ophthalmic, nasal, transdermal or parenteral route. A preferred route is oral administration. The present invention notably covers the use of a compound according to the present invention for the manufacture of pharmaceutical composition.

Such medicament can take the form of a pharmaceutical composition adapted for oral administration, which can be formulated using pharmaceutically acceptable carriers well known in the art in suitable dosages. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

The composition of the invention can also take the form of a pharmaceutical composition for topical administration.

Such compositions may be presented in the form of a gel, paste, ointment, cream, lotion, liquid suspension aqueous, aqueous-alcoholic or, oily solutions, or dispersions of the lotion or serum type, or anhydrous or lipophilic gels, or emulsions of liquid or semi-solid consistency of the milk type, obtained by dispersing a fatty phase in an aqueous phase or vice versa, or of suspensions or emulsions of soft, semi-solid consistency of the cream or gel type, or alternatively of microemulsions, of microcapsules, of microparticles or of vesicular dispersions to the ionic and/or nonionic type.

The composition according to the invention comprises any ingredient commonly used in dermatology and cosmetic. It may comprise at least one ingredient selected from hydrophilic or lipophilic gelling agents, hydrophilic or lipophilic active agents, preservatives, emollients, viscosity enhancing polymers, humectants, surfactants, preservatives, antioxidants, solvents, and fillers, antioxidants, solvents, perfumes, fillers, screening agents, bactericides, odor absorbers and coloring matter.

As oils which can be used in the invention, mineral oils (liquid paraffin), vegetable oils (liquid fraction of shea butter, sunflower oil), animal oils, synthetic oils, silicone oils (cyclomethicone) and fluorinated oils may be mentioned. Fatty alcohols, fatty acids (stearic acid) and waxes (paraffin, carnauba, beeswax) may also be used as fatty substances.

As emulsifiers which can be used in the invention, glycerol stearate, polysorbate 60 and the PEG-6/PEG-32/glycol stearate mixture are contemplated. As hydrophilic gelling agents, carboxyvinyl polymers (carbomer), acrylic copolymers such as acrylate/alkylacrylate copolymers, polyacrylamides, polysaccharides such as hydroxypropylcellulose, clays and natural gums may be mentioned, and as lipophilic gelling agents, modified clays such as bentones, metal salts of fatty acids such as aluminum stearates and hydrophobic silica, or alternatively ethylcellulose and polyethylene may be mentioned.

As hydrophilic active agents, proteins or protein hydrolysates, amino acids, polyols, urea, allantoin, sugars and sugar derivatives, vitamins, starch and plant extracts, in particular those of Aloe vera may be used.

As lipophilic active, agents, retinol (vitamin A) and its derivatives, tocopherol (vitamin E) and its derivatives, essential fatty acids, ceramides and essential oils may be used. These agents add extra moisturizing or skin softening features when utilized.

In addition, a surfactant can be included in the composition so as to provide deeper penetration of the compound capable of depleting mast cells, such as a tyrosine kinase inhibitor.

Among the contemplated ingredients, the invention embraces penetration enhancing agents selected for example from the group consisting of mineral oil, water, ethanol, triacetin, glycerin and propylene glycol; cohesion agents selected for example from the group consisting of polyisobutylene, polyvinyl acetate and polyvinyl alcohol, and thickening agents.

Chemical methods of enhancing topical absorption of drugs are well known in the art. For example, compounds with penetration enhancing properties include sodium lauryl sulfate (Dugard, P. H. and Sheuplein, R. J., “Effects of Ionic Surfactants on the Permeability of Human Epidermis: An Electrometric Study,” J. Ivest. Dermatol., V. 60, pp. 263-69, 1973), lauryl amine oxide (Johnson et. al., U.S. Pat. No. 4,411,893), azone (Rajadhyaksha, U.S. Pat. Nos. 4,405,616 and 3,989,816) and decylmethyl sulfoxide (Sekura, D. L. and Scala, J., “The Percutaneous Absorption of Alkylmethyl Sulfides,” Pharmacology of the Skin, Advances In Biolocy of Skin, (Appleton-Century Craft) V. 12, pp. 257-69, 1972). It has been observed that increasing the polarity of the head group in amphoteric molecules increases their penetration-enhancing properties but at the expense of increasing their skin irritating properties (Cooper, E. R. and Berner, B., “Interaction of Surfactants with Epidermal Tissues: Physiochemical Aspects,” Surfactant Science Series, V. 16, Reiger, M. M. ed. (Marcel Dekker, Inc.) pp. 195-210, 1987).

A second class of chemical enhancers are generally referred to as co-solvents. These materials are absorbed topically relatively easily, and, by a variety of mechanisms, achieve permeation enhancement for some drugs. Ethanol (Gale et al., U.S. Pat. No. 4,615,699 and Campbell et al., U.S. Pat. Nos. 4,460,372 and 4,379,454), dimethyl sulfoxide (U.S. Pat. No. 3,740,420 and U.S. Pat. No. 3,743,727, and U.S. Pat. No. 4,575,515), and glycerine derivatives (U.S. Pat. No. 4,322,433) are a few examples of compounds which have shown an ability to enhance the absorption of various compounds.

The pharmaceutical compositions of the invention can also be intended for administration with aerosolized formulation to target areas of a patient's respiratory tract.

Devices and methodologies for delivering aerosolized bursts of a formulation of a drug is disclosed in U.S. Pat. No. 5,906,202. Formulations are preferably solutions, e.g. aqueous solutions, ethanoic solutions, aqueous/ethanoic solutions, saline solutions, colloidal suspensions and microcrystalline suspensions. For example aerosolized particles comprise the active ingredient mentioned above and a carrier, (e.g., a pharmaceutically active respiratory drug and carrier) which are formed upon forcing the formulation through a nozzle which nozzle is preferably in the form of a flexible porous membrane. The particles have a size which is sufficiently small such that when the particles are formed they remain suspended in the air for a sufficient amount of time such that the patient can inhale the particles into the patient's lungs.

The invention encompasses the systems described in U.S. Pat. No. 5,556,611:

liquid gas systems (a liquefied gas is used as propellent gas (e.g. low-boiling FCHC or propane, butane) in a pressure container,

suspension aerosol (the active substance particles are suspended in solid form in the liquid propellent phase),

pressurized gas system (a compressed gas such as nitrogen, carbon dioxide, dinitrogen monoxide, air is used.

Thus, according to the invention the pharmaceutical preparation is made in that the active substance is dissolved or dispersed in a suitable nontoxic medium and said solution or dispersion atomized to an aerosol, i.e. distributed extremely finely in a carrier gas. This is technically possible for example in the form of aerosol propellent gas packs, pump aerosols or other devices known per se for liquid misting and solid atomizing which in particular permit an exact individual dosage.

Therefore, the invention is also directed to aerosol devices comprising the compound as defined above and such a formulation, preferably with metered dose valves.

The pharmaceutical compositions of the invention can also be intended for intranasal administration.

In this regard, pharmaceutically acceptable carriers for administering the compound to the nasal mucosal surfaces will be readily appreciated by the ordinary artisan. These carriers are described in the Remington's Pharmaceutical Sciences” 16th edition, 1980, Ed. By Arthur Osol, the disclosure of which is incorporated herein by reference.

The selection of appropriate carriers depends upon the particular type of administration that is contemplated. For administration via the upper respiratory tract, the composition can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or upper respiratory administration.

Common intranasal carriers include nasal gels, creams, pastes or ointments with a viscosity of, e.g., from about 10 to about 3000 cps, or from about 2500 to 6500 cps, or greater, may also be used to provide a more sustained contact with the nasal mucosal surfaces. Such carrier viscous formulations may be based upon, simply by way of example, alkylcelluloses and/or other biocompatible carriers of high viscosity well known to the art (see e.g., Remington's, cited supra. A preferred alkylcellulose is, e.g., methylcellulose in a concentration ranging from about 5 to about 1000 or more mg per 100 ml of carrier. A more preferred concentration of methyl cellulose is, simply by way of example, from about 25 to about mg per 100 ml of carrier.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. For nasal administration of solutions or suspensions according to the invention, various devices are available in the art for the generation of drops, droplets and sprays.

A premeasured unit dosage dispenser including a dropper or spray device containing a solution or suspension for delivery as drops or as a spray is prepared containing one or more doses of the drug to be administered and is another object of the invention. The invention also includes a kit containing one or more unit dehydrated doses of the compound, together with any required salts and/or buffer agents, preservatives, colorants and the like, ready for preparation of a solution or suspension by the addition of a suitable amount of water.

The invention is explained below with more details:

Masitinib has the following formula:

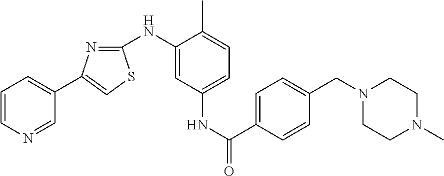

This compound is also known under reference AB1010.

The drug product is a tyrosine kinase inhibitor developed by AB Science that does not yet have a designated trade name, but referred to as AB1010. In this document we refer to the drug product by the name of its active pharmaceutical ingredient, masitinib. For human clinical trials all doses are expressed in terms of masitinib (molecular formula: C₂₈H₃₀N₆OS; relative molecular mass of 498.7), which is also known as the free base of masitinib. The investigational medicinal product contains the mesylate salt of masitinib, which is also known as masitinib mesylate (molecular formula: C₂₉H₃₄N₆O₄S₂; relative molecular mass of 549.8). The use of a salt form provides a good solubility and drug bioavailability. According to the invention the mesylate salt of masitinib is preferred.

### Masitinib has a Higher Affinity for the Targets Specific to GIST (Kit WT or Mutated and PDGFRA)

Pre-clinical studies have shown that masitinib is a potent inhibitor of the targets specific to GIST, with better affinity than imatinib [7] which is incorporated herein by reference in its entirety. A summary of the results is presented in Table 1 below:

TABLE 1
In vitro inhibitory properties of masitinib and imatinib on cell
proliferation

| Cell proliferation assay | | | |
|---|---|---|---|
| (IC₅₀) | masitinib | imatinib | |
| Human Kit WT | 150 nM | 100-200 | nM |
| Human Kit exon 9 | 100 nM | ~200 | nM |
| Human Kit exon 11 | 3 nM | 27 | nM |
| Human Kit exon 13 | 40 nM (GIST 882 cells) | 120 | nM |
| | | | (GIST |
| | | | 882 cells) |
| PDGFRA | 250 nM | 1 | μM |

Masitinib is Efficient Against Cell Lines that are Rendered Resistant to Imatinib

HMC-1α155 cells (a human mast cell line expressing Kit with the mutation V560G) were rendered resistant to imatinib by exposing them to 1 μM imatinib until exponential growth was observed, indicating that the cells had become resistant. HMC-1 is a human mast cell line derived from a patient with mast cell leukaemia. The cell line used in this study, HMC-1α155, is a clone derived from the original population expressing endogenous Kit bearing the mutation Val 560 to Gly. Cells were placed in a medium containing 0.2 and 1 μM of AB1010 or imatinib. The medium used in this case was a RPMI medium containing L-glutamine (Cambrex cat#12-702F) supplemented with 100 U/mL penicillin and 100 μg/mL streptomycin (Cambrex 100× penicilline/streptomycine mixture cat #17-602E), and with 10% v/v foetal calf serum (AbCys Lot S02823S1800) which has been previously heat-inactivated 30 minutes at 56° C. (RPMI 10). The medium containing the drugs was replaced with fresh one every 3-4 days. The cells were maintained in these conditions for several weeks and until exponential growth was observed, indicating that the cells had become drug resistant. Resistant cells lines were then tested for their sensitivity to masitinib and imatinib using apoptosis assay [8]. The principle behind the method for apoptosis assay by Propidium Iodide Staining (PI staining) is as follows: during apoptosis, DNA breakup causes small fragments of DNA to be free in the nucleus. Following appropriate elution with citrate buffer, these fragments are lost from the nucleus. As these cells now have a lower DNA content, subsequent staining with a DNA binding dye will reveal these cells in the sub-G1 region.

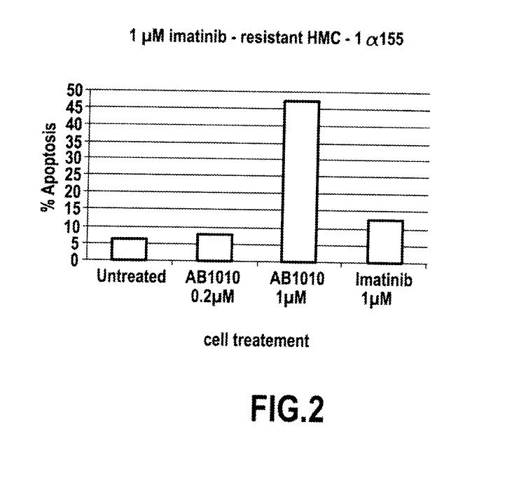

More than 45% of imatinib-resistant HMC-1α155 cells were in apoptosis when exposed to 1 μM masitinib (FIG. 2). Thus imatinib-resistant cells significantly retain sensitivity to masitinib.
The % apoptosis represents the percentage of cells in apoptose with respect to overall cells.
Masitinib has Anti-Metastatic Properties Possibly Due to its Interaction with the FAK Pathway

Data from pre-clinical and clinical studies both in dogs and humans suggests that masitinib reduces the number of patients developing metastases while under treatment. Since masitinib has been shown to reduce FAK activity, and since FAK has been involved in cell proliferation and migration, it is thought that this reduced risk to develop metastases under masitinib could be due to its action on FAK pathway.

In vitro, masitinib mesilate potently inhibits the GIST related c-Kit gain-of-function mutant V559D (exon 11), (Dubreuil et al., 2009-[7]). However, the extent of long-term survival observed in patients treated with masitinib, especially in comparison to other tyrosine kinase inhibitors (e.g. imatinib), far exceeds expectations. That is to say, this gain in efficacy cannot be explained solely by masitinib's superior inhibition of c-Kit, or inhibition of other individual kinase targets. Surprisingly, it would seem, without wishing to be bound by the theory, that concomitant processes contribute to masitinib's efficacy in GIST including, but not restricted to: masitinib's anti-mastocyte activity through targeting wild-type c-Kit, and indirectly therefore inhibition of the array of mediators they release; inhibition of mast cell degranulation through Lyn and Fyn inhibition, key components of the transduction pathway leading to mast cell IgE induced degranulation; inhibition of the FAK pathway; down-regulation of the Wnt/β-catenin signalling pathway. Thus, masitinib appears to exert an anticancer (GIST) action that extends beyond its inherent tyrosine kinase inhibition profile.

In the drawings:

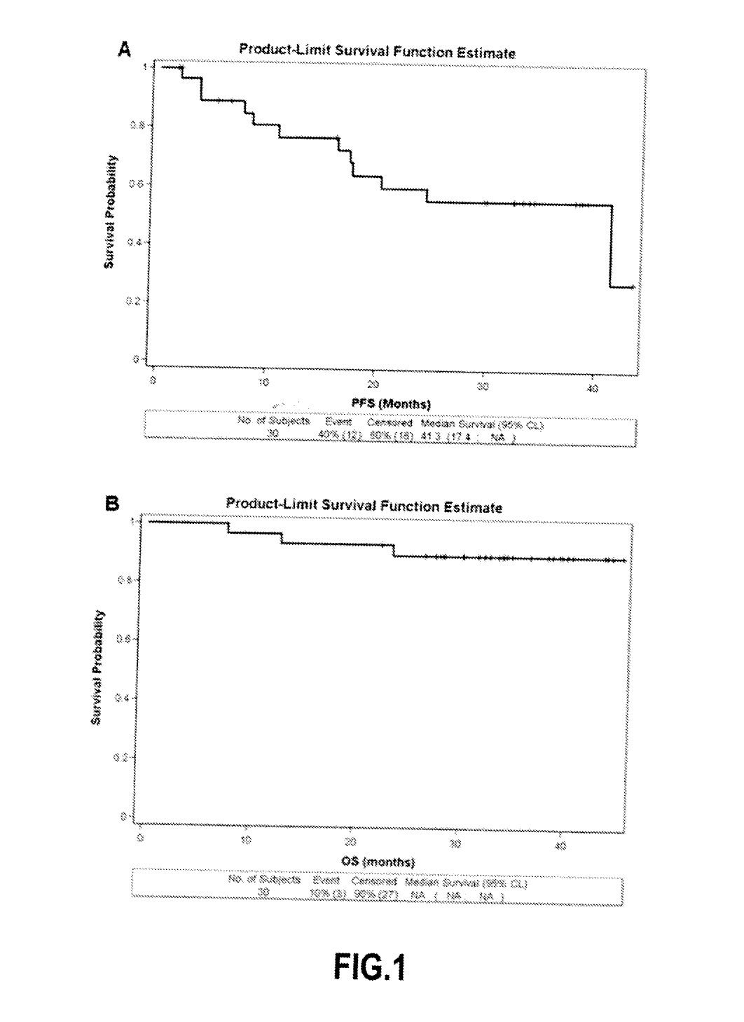

FIG. 1: Kaplan-Meier analyses of progression-free survival (A) and overall survival (B).

FIG. 2: Diagram showing results of the % apoptosis by different KIT inhibitors (AB1010 and imatinib) versus control (untreated) on cells resistant to imatinib.

### EXAMPLES

The following example illustrates the invention, but is not, however, intended to limit the scope of the invention in any way. Other test models known as such to the person skilled in the pertinent art can also determine the beneficial effects of the use of masitinib mesylate, or salts thereof.

An open-label, multicenter, non-randomised, phase 2 clinical trial was conducted to evaluate the efficacy and safety of masitinib mesylate in patients with advanced GIST.

Methods; Patients: Patients enrolled in this study were aged over 18 years with inoperable, non-pretreated, histologically proven locally advanced or metastatic, c-Kit positive GIST. Each patient had measurable tumour lesions according to response evaluation criteria in solid tumours (RECIST)[9] and an Eastern Cooperative Oncology Group (ECOG) performance status of ≦2. Exclusion criteria included: inadequate organ function defined via blood tests, severe liver or cardiac failure, and severe neurological or psychiatric disorders. Patients receiving a concomitant treatment within 4 weeks before inclusion, and pregnant or lactating women were also excluded.

Methods; Treatment: oral masitinib, supplied as 100 and 200 mg tablets, was administered daily at 7.5 mg/kg/day, in two intakes during meals. The 30 patients included received a mean dose of 7.1±0.8 mg/kg/day of masitinib; the median dose was 7.2 mg/kg/day; the range was 3.5 to 8.7 mg/kg/day; Q1 and Q3 were 6.9 and 7.6 mg/kg/day, respectively. This does not deviate significantly from the dose of 7.5 mg/kg/day planned by the protocol.

The chemical name is 4-(4-methylpiperazin-1-ylmethyl)-N-[4-methyl-3-(4-pyridin-3-ylthiazol-2-ylamino) phenyl]benzamide-mesylate methane sulfonic acid salt, and the chemical formula is C₂₈H₃₀N₆OS.CH₄O3S. Masitinib used in these studies was synthesised by either Archemis (Decines Charpieu, (France) or Syngene-Biocon (Bangalore, India). For detailed procedure refer to patent WO/2008/098949. AB1010 is also manufactured by AB Science, S.A. (France), or by Prestwick Chemical, Inc. (France). The chemical structure was confirmed by nuclear magnetic resonance, mass spectrometry, ultraviolet and infrared spectrometry, and elemental analysis. Masitinib is practically insoluble, in 0.1M NaOH and n-hexane, slightly soluble in ethanol and propylene glycol, soluble in water, and freely soluble in 0.1M HCl and dimethylsulfoxide. The compound, a white powder, was prepared according to the art to provide tablets. Tablet containing masitinib was used for each experiment.

Methods; Efficacy and Safety assessment: safety was assessed for all patients receiving at least one dose of masitinib with toxicity graded according to the NCl CTCAE v3.0 classification. All adverse events (AEs), including abnormal serology or haematology, were recorded regardless of causality.

The primary efficacy endpoint was response rate (RR) after 2 months of masitinib treatment according to RECIST, using Computed Tomography (CT). Secondary efficacy endpoints were the evaluation of metabolic response [10] using [¹⁸F]-fluorodeoxyglucose-Positron Emission Tomography (FDG-PET) and assessment of disease control rate, PFS and OS. For each patient, all efficacy parameters were recorded on the first day of treatment (baseline) prior to administration of masitinib, then on weeks 2, 4, 8, and 16, and every 12 weeks thereafter (extension phase).

Time-to-event analyses were calculated from the date of first masitinib intake to the date of event (documented progression or death). Patients who had not progressed at the date of last tumor assessment were censored at that date for PFS. Patients alive at the time of the analysis were censored at the date of last contact for OS.

Methods; Statistical analyses: Simon minimax two-stage design was used for this prospective, multicenter, single-group, phase II trial. Fourteen patients were initially enrolled, with recruitment of an additional ten patients being dependent on the occurrence of at least one objective RECIST response. This population was further extended to a total of 30 patients to ensure a sufficient evaluable population for all end-points; the type I(α) error was 5% (two-sided) for all analyses. Quantitative variables were described by the number of filled and missing data, mean, standard deviation, median, minimum and maximum. Qualitative variables were described by the number of missing data and, for each modality, frequency and percentage (referring to filled data). Time-to-event data were described using Kaplan-Meier (KM) estimates. The median was provided with its 95% confidence interval (95% CI). PFS rates were given every 6 months with KM estimates. All data analyses and reporting procedures used SAS v9.1 in a Windows XP operating system environment.

Results; Patient characteristics: between June 2005 and April 2007, 30 patients were enrolled from five centres across France. Patient characteristics at inclusion are summarized in Table 2. At the cut-off date of April 2009, the median follow-up duration was 33.7 months (range 7.7-45.4 months). All analyses are reported for the intent-to-treat population (ITT), defined as all enrolled patients (N=30). Two protocol deviations were revealed following pathology review, with two patients having been misdiagnosed as having GIST; one had a low grade endometrial stromal tumour and the other had an aggressive fibromatosis.

Four patients terminated prematurely before the fourth month: one patient for progressive disease (PD), one on the investigator's decision and two patients for AEs (non treatment-related grade 3 paresis; and treatment-related, grade 3 cheilitis and skin toxicity). During the extension period, 13 patients terminated the study: one patient for protocol violation (endometrial stromal tumour), eight patients for disease progression, two patients for AEs (one died from non treatment-related post-surgical complication and the other had treatment-related, grade 3 psoriasis), and two patients for other reasons (one patient developed a metastatic prostate cancer necessitating systemic chemotherapy and one discontinued therapy after radiofrequency ablation of liver metastasis).

TABLE 2
Demographics and clinical characteristics of patients

| Parameter | ITT Population (N = 30) | |
|---|---|---|
| Age (years) | Mean ± SD | 57 ± 14 |
| | Median | 58 |
| | Range | 34-82 |
| Sex, N (%) | Female | 12 (40%) |
| | Male | 18 (60%) |
| Weight (kg) | Mean ± SD | 75 ± 15 |
| | Median | 75 |
| | Range | 51-115 |
| ECOG performance status, N (%) | 0 | 23 (77%) |
| | 1 | 7 (23%) |
| Previous treatments/medication for GIST | Surgery | 21 (70%) |
| | Biopsy | 4 (13%) |
| | Other | 2 (7%) |
| c-Kit status | Positive | 29 (96.7%) |
| | Negative | 1 (3.3%) |
| Time since diagnosis (months) | Mean ± SD | 22 ± 28 |
| | Median | 13 |
| | Range | 0-131 |

Results; Safety assessment: Safety analyses were performed on the ITT population (Table 3). All patients reported at least one treatment-related AE; 14/30 patients (47%) experienced at least one grade 3 treatment-related AE, of which rash was the most frequent at 3/30 patients (10%); and 1/30 patient (3.3%) reported one grade 4 AE (skin exfoliation). A total of 14 serious adverse events (SAE) were experienced by 8/30 patients (27%), three of which were treatment-related (worsening of a concomitant psoriasis and anaemia). The most frequent treatment-related toxicities per patient were: asthenia (83%), diarrhoea (57%), eye oedema (47%), nausea (47%), muscle spasms (40%), cutaneous rash (40%), abdominal pain (33%), pruritus (33%), vomiting (23%), upper abdominal pain (23%) and peripheral oedema (20%). Treatment-related oedemas (all types) were experienced by 21/30 patients (70%).

Six patients (20%) had their dose reduced by 100 or 200 mg/day (three patients each), following grade 3-4 AEs, and 16/30 patients (53.3%) had treatment interruption for more than 8 days. Reasons for treatment interruptions were non-haematological AEs for 13/30 patients (43%) (treatment-related for twelve of them), treatment-related haematological toxicity for 1/30 patient (3%), and surgery for 2/30 patients (6.7%). The most frequent treatment-related, non-haematological AEs leading to interruptions were skin toxicity, oedema and asthenia. Thirteen out of thirty patients (43%) were still undergoing treatment with masitinib at the cut-off date (12 at the same initial dose), with treatment duration from 26.5 to 45.4 months.

TABLE 3
Frequent adverse events (>10%) in patients receiving masitinib, and
their suspected relationship to masitinib

| Number (%) | | | | |
|---|---|---|---|---|
| of patients | Suspected* | | All causalities | |
| (N = 30) | All grades | G3 + G4 | All grades | G3 + G4 |
| Haematological | | | | |
| events | | | | |
| Anaemia | 4 (13.3%) | 1 (3.3%) | 6 (20.0%) | 1 (3.3%) |
| Neutropenia | 5 (16.7%) | 2 (6.7%) | 5 (16.7%) | 2 (6.7%) |
| Non- | | | | |
| haematological | | | | |
| events | | | | |
| Asthenia | 25 (83.3%) | 1 (3.3%) | 27 (90.0%) | 1 (3.3%) |
| Diarrhoea | 17 (56.7%) | 1 (3.3%) | 18 (60.0%) | 1 (3.3%) |
| Abdominal | 10 (33.3%) | 1 (3.3%) | 16 (53.3%) | 2 (6.7%) |
| Pain | | | | |
| Nausea | 14 (46.7%) | | 15 (50.0%) | |
| Eye Oedema | 14 (46.7%) | 1 (3.3%) | 14 (46.7%) | 1 (3.3%) |
| Muscle Spasms | 12 (40.0%) | | 12 (40.0%) | |
| Rash | 12 (40.0%) | 3 (10.0%) | 12 (40.0%) | 3 (10.0%) |
| Pruritus | 10 (33.3%) | 1 (3.3%) | 11 (36.7%) | 1 (3.3%) |
| Vomiting | 7 (23.3%) | | 10 (33.3%) | |
| Abdominal | 7 (23.3%) | | 9 (30.0%) | |
| Pain Upper | | | | |
| Oedema | 6 (20.0%) | | 8 (26.7%) | |
| Peripheral | | | | |
| Eyelid Oedema | 7 (23.3%) | | 7 (23.3%) | |
| Erythema | 5 (16.7%) | | 6 (20.0%) | |
| Mucosal | 5 (16.7%) | 1 (3.3%) | 5 (16.7%) | 1 (3.3%) |
| Inflammation | | | | |
| Dry Skin | 4 (13.3%) | | 4 (13.3%) | |
| Lacrimation | 4 (13.3%) | | 4 (13.3%) | |
| Increased | | | | |
| Myalgia | 4 (13.3%) | | 4 (13.3%) | |

*Suspected: treatment related or not assessable; G3: grade 3 AE; G4: grade 4 AE.

Results; Response to treatment: during the Simon first stage, 4/14 patients had a confirmed PR after 2 months of treatment, instigating the study's Simon second stage. Efficacy results are presented in Table 4. Among the ITT population there were: 6/30 PR (20%), 23/30 SD (76.7%) and 1/30 PD (3.3%) after 2 months of masitinib treatment. Best response (RECIST) was analyzed until the cut-off date: complete response (CR), PR, SD and PD were recorded for 1/30 (3.3%), 15/30 (50%), 13/30 (43.3%), and 1/30 (3.3%) patients, respectively. The overall response rate (CR+PR) was 16/30 (53.3%) patients (95% CI [34.3; 71.7]) with a disease control rate (CR+PR+SD) of 29/30 (96.7%) patients (95% CI [82.8; 99.9]). Median time to first objective response was 5.6 months (range: 0.8-23.8 months).

Metabolic response was assessed for 17/30 patients (56.7%), of which 3/30 patients (10%) had a negative FDG-PET at baseline. Of the 13/30 (43.3%) and 14/30 patients (47.7%) who were evaluable after 1 and 2 months of treatment, respectively: 9/13 (69.2%) had a partial metabolic response (PMR) and 4/13 (30.8%) had a stable metabolic disease (SMD) after 1 month; whilst 3/14 (21.4%) had a complete metabolic response (CMR), 9/14 (64.3%) had a PMR, and 2/14 (14.3%) had a SMD, after 2 months. The metabolic response rate (CMR+PMR) after 2 months of treatment was 12/14 (85.7%) patients (95% CI [57.2; 98.2]).

TABLE 4
Response rates

| Response | | | | |
|---|---|---|---|---|
| (RECIST); n (%) | 2 months (N = 30) | | Best Response (N = 30) | |
| CR | 0 | (0.0%) | 1 | (3.3%) |
| PR | 6 | (20.0%) | 15 | (50.0%) |
| CR + PR [95% CI] | 6 | (20.0%) [7.7; 38.6] | 16 | (53.3%) [34.3; 71.7] |
| SD | 23 | (76.7%) | 13 | (43.3%) |
| CR + PR + SD | 29 | (96.7%) [82.8; 99.9] | 29 | (96.7%) [82.8; 99.9] |
| [95% CI] | | | | |
| PD | 1 | (3.3%) | 1 | (3.3%) |
| Metabolic response | At 1 month (N = 13) | | At 2 months (N = 14) | |
| CMR | 0 | (0.0%) | 3 | (21.4%) |
| PMR | 9 | (69.2%) | 9 | (64.2%) |
| CMR + PMR | 9 | (69.2%) | 12 | (85.7%) |
| [95% CI] | | [38.6-90.9] | | [57.2-98.2] |
| SMD | 4 | (30.8%) | 2 | (14.3%) |

CR: complete response,
PR: partial response;
CR + PR: overall response rate;
SD: stable disease;
CR + PR + SD: disease control rate;
PD: progressive disease;
CMR: complete metabolic response,
PMR: partial metabolic response;
CMR + PMR: metabolic response rate;
SMD: stable metabolic disease.

Results; Time-to-event analysis: the analysis revealed 12 events (11 progressions and one death) with 18/30 patients (60%) censored for PFS: six patients withdrew from the study without progression and 12 progression-free patients were still receiving masitinib at the cut-off date. The estimated 6-month, 1-year, 2-year and 3-year PFS rates were 88.9% (95% CI [69.4; 96.3]), 76.8% [55.3; 88.9], 59.7% [37.9; 76.0] and 55.4% [33.9; 72.5], respectively (Table 5). Median PFS was 41.3 months [17.4 months; not reached] according to KM analysis (FIG. 1A). Median OS was not reached (FIG. 1B and Table 5), with 1-year survival rate of 96.7% [78.6; 99.5], and 2- and 3-year survival rates each at 89.9% [71.8; 96.6].

TABLE 5
PFS, PFS rates, OS and OS rates

| PFS | |
|---|---|
| Median | 41.3 months |
| [95% CI] | [17.4-NR] |
| PFS rate (%) [95% CI] | |
| 6 months | 88.9 [69.4; 96.3] |
| 12 months | 76.8 [55.3; 88.9] |
| 18 months | 64.0 [42.0; 79.5] |
| 24 months | 59.7 [37.9; 76.0] |
| 30 months | 55.4 [33.9; 72.5] |
| 36 months | 55.4 [33.9; 72.5] |
| 42 months | 27.7 [2.0; 65.7] |
| OS | |
| Median | NR |
| [95% CI] | [NR; NR] |
| OS rates (%) [95% CI] | |
| 12 months | 96.7 [78.6; 99.5] |
| 24 months | 89.9 [71.8; 96.6] |
| 36 months | 89.9 [71.8; 96.6] |

PFS: progression-free survival;
OS: overall survival;
NR: not reached.

Results; Mutational analysis: biopsy material was collected from 29/30 patients (96.7%) to assess their c-Kit status. Sufficient biopsy material was available to perform mutational analysis for 15/30 patients (50%): 10/30 patients (33.3%) had a GIST harbouring a c-Kit exon 11 mutation, 1/30 patient (3.3%) had double c-Kit exon 11 and 13 mutations, 3/30 patients (10%) had a WT c-Kit, and 1/30 patient (3.3%) had a GIST harbouring the PDGFRα (or PDGFRA) mutation (D842V).

Discussion; Imatinib has dramatically improved the outcome of patients with advanced GIST, becoming the model for targeted therapy in solid tumours [11-13]. However, despite near optimal compliance in the majority of patients and extended administration of imatinib [14], the risk of secondary progression due to acquired resistance to imatinib persists over time [15, 16]. This highlights the need for new strategies in non-pre-treated advanced GIST to increase the rate of complete remission and the duration of progression arrest rate.

It has been shown that some patients benefit from a higher than the standard imatinib dose, suggesting that individualized treatment could be a critical option in the initial management of advanced GIST patients. This is evidenced by imatinib at 800 mg/day producing improved PFS, as compared to the standard dose of 400 mg/day [17], in patients whose GIST harbours an exon 9 mutation [18, 19]; the relationship between imatinib plasma levels and progression [6]; and the fact that one third of patients progressing under imatinib at 400 mg/day clearly benefited from the higher dose regimen [13, 20]. In contrast to imatinib's fixed dosing strategy, masitinib has been developed with patient weight-adjusted dosing in mind [21]. Given its higher selectivity for c-Kit [7], a patient-optimized dose of masitinib could possibly provide a significant therapeutic benefit; although dose increments smaller than the 100 mg used in for this study are likely to be required to achieve such optimization.

As expected with the selectivity profile of masitinib [7], no cardiac side-effects have been observed to date. Occurrences of the most common masitinib-related haematological AEs (neutropenia and anaemia) were substantially lower compared to imatinib at standard dose [12]. The most frequently reported masitinib-related, non-haematological AEs were similar to those reported with imatinib in front-line treatment, with the exception of rash and abdominal pain that occurred at a higher frequency for masitinib [12]. In general, AEs occurred early during the course of treatment, which is consistent with the known safety profile of tyrosine kinase inhibitors [22, 23]; the majority of AEs showing a clear decrease in frequency for the 24/30 patients (80%) treated beyond 6 months (data not shown). The implication here is that treatment tolerance is likely to improve after the initial 6 months, thereby, making masitinib more appropriate for any long-term treatment regimen. At the cut-off date, 12/30 patients (40%) were still receiving masitinib at the same initial daily dose.

Early resistance to imatinib has been defined as progression occurring within the first 6 months of treatment in patients who showed no response. It is observed in 10-15% of patients and appears to result from intrinsic factors present before treatment start [16]. In this study only 1/30 patient (3.3%) never benefited from masitinib, suggesting that masitinib may be less susceptible to early resistance; although further investigation is required to confirm this hypothesis.

The objective response (RECIST) and metabolic response rate at 2 months are in the range of those observed with imatinib [12, 24]. Combinations of morphologic (Computed Tomography) and functional imaging techniques such as FDG-PET or Dynamic Contrast Enhanced-Ultrasonography (DCE-US) [25] highlight again the discrepancy between the biological (cellular level) and clinical (radiological level) activities of TKIs in GIST [11, 26, 27]. As observed with imatinib, masitinib induces changes in the tumour structure, such as decreased tumour vascularity, haemorrhage or necrosis, cystic or myxoid degeneration, that are consistent with a therapeutic activity with or without a change in tumour volume. When these three different radiological tumour assessments were applied to the same patients, masitinib was found to induce tumour response in only 20% of evaluable patients according to changes in tumour size (RECIST) but in 86% of patients according to metabolic response using FDG-PET and in 75% of patients assessed with DCE-US [28], after 2 months of masitinib. Interestingly, one patient with an FDG-PET CMR observed after 2 months of masitinib had a decrease but not a disappearance of contrast uptake with DCE-US performed concomitantly, suggesting that this less expensive tool assessing both tumour size and structure may be a more reliable measure of the residual activity of GIST tumour cells than FDG-PET. As for imatinib [25] and other TKIs [28], a decrease of contrast uptake assessed with DCE-US, 7 and 14 days after the beginning of masitinib, correlates with a good response on CT scan at 2 months [28].

As already reported, RECIST is not optimal for an early response assessment of c-Kit inhibitors in GIST patients[29] since the pattern of radiological response has no prognostic value for further outcome, except for PD [30]. However, RECIST assessment can be used for practical decision making since absence of progression according to RECIST turned out to be an excellent predictive marker of benefit with masitinib in terms of PFS. Consequently, masitinib needs to be continued as long as there is no progression according to RECIST; an absence of tumour progression under masitinib being equivalent to tumour response.

Twelve of the 16 patients who withdrew from the study (eight for PD, three for AEs, and one on the investigator's decision) were switched to imatinib-treatment. Of the eight patients progressing under masitinib, six received imatinib at 800 mg/day and two received imatinib at 400 mg/kg/day, with a median exposure of 5.4 months. Six of these patients discontinued imatinib for AEs or progression, the remainder (one at each dose level) showed some relevant disease stabilization. This suggests that the use of a less selective c-Kit inhibitor (i.e. imatinib) in second line therapy precludes any relevant activity in terms of tumour volume reduction and that therefore, patients progressing under masitinib are candidates for alternative second-line targeted therapies [31]. Of those patients intolerant to masitinib; one died, one had PD and switched to an alternate second line therapy, and the other showed a PR.

This study was designed to assess the objective response rate according to RECIST at 2 months under masitinib, although the time to secondary resistance to masitinib (i.e. PFS) would have been a more relevant activity screening end-point (as with imatinib or sunitinib). Despite this study's small number of patients (with a majority of GIST harbouring a c-Kit exon 11 mutation), a median follow-up of 33.7 months and the limited validity of comparison with phase III trials; the median PFS (41.3 months), as well as the 2- and 3-year PFS rates (60% and 55%, respectively) observed with masitinib, compare favourably with those of imatinib at 400 mg/day [5, 12].

In summary, the activity of masitinib in GIST could in part be due to: (1) its potent inhibition of WT and JM c-Kit that limits tumour proliferation and emergence of resistant cell clones; (2) its partial inhibition of the FAK pathway that may limit the development of metastases, thus, slowing down progression [32]; and (3) individual adaptation of the daily dose that may offer an optimal dose over time. Indications that masitinib provides sustainable benefits, as evidenced by the 2 and 3 year OS data, are promising, but its impact on OS has to be further determined with a follow-up of those responding patients (43%) still receiving treatment, as well as progressing patients who went on to receive alternative treatments [5].

Conclusion; Results from this study help to further establish the therapeutic role of TKIs that selectively inhibit c-Kit [5, 11-13]. Moreover, within the limitations of an uncontrolled phase 2 trial, this study shows that masitinib may offer an effective and relatively well-tolerated treatment for non-pre-treated, inoperable, locally advanced or metastatic GIST patients. Confirmatory phase III trials comparing masitinib to imatinib in first-line treatment will confirm the validity of these findings and help to further investigate the long-term efficacy and safety of masitinib.

### BIBLIOGRAPHY INCORPORATED HEREIN BY REFERENCE

1. Hirota, S., et al., Gain-of-function mutations of c-kit in human gastrointestinal stromal tumors. Science, 1998. 279(5350): p. 577-80.
2. Kindblom, L. G., et al., Gastrointestinal pacemaker cell tumor (GIPACT): gastrointestinal stromal tumors show phenotypic characteristics of the interstitial cells of Cajal. Am J Pathol, 1998. 152(5): p. 1259-69.
3. Heinrich, M. C., et al., PDGFRA activating mutations in gastrointestinal stromal tumors. Science, 2003. 299(5607): p. 708-10.
4. Sleijfer, S., et al., Improved insight into resistance mechanisms to imatinib in gastrointestinal stromal tumors: a basis for novel approaches and individualization of treatment. Oncologist, 2007. 12(6): p. 719-26.
5. Blanke, C. D., et al., Long-term results from a randomized phase II trial of standard-versus higher-dose imatinib mesylate for patients with unresectable or metastatic gastrointestinal stromal tumors expressing KIT. J Clin Oncol, 2008. 26(4): p. 620-5.
6. Demetri, G. D., et al., Imatinib plasma levels are correlated with clinical benefit in patients with unresectable/metastatic gastrointestinal stromal tumors. J Clin Oncol, 2009. 27(19): p. 3141-7.
7. Dubreuil, P., et al., Masitinib (AB1010), a potent and selective tyrosine kinase inhibitor targeting KIT. PLoS ONE, 2009. 4(9): p. e7258.
8. Darzynkiewicz, Z., et al., Features of apoptotic cells measured by flow cytometry. Cytometry, 1992. 13(8): p. 795-808.
9. Therasse, P., et al., New guidelines to evaluate the response to treatment in solid tumors. European Organization for Research and Treatment of Cancer, National Cancer Institute of the United States, National Cancer Institute of Canada. J Natl Cancer Inst, 2000. 92(3): p. 205-16.
10. Jager, P. L., J. A. Gietema, and W. T. van der Graaf, Imatinib mesylate for the treatment of gastrointestinal stromal tumours: best monitored with FDG PET. Nucl Med Commun, 2004. 25(5): p. 433-8.
11. Demetri, G. D., et al., Efficacy and safety of imatinib mesylate in advanced gastrointestinal stromal tumors. N Engl J Med, 2002. 347(7): p. 472-80.
12. Verweij, J., et al., Progression-free survival in gastrointestinal stromal tumours with high-dose imatinib: randomised trial. Lancet, 2004. 364(9440): p. 1127-34.
13. Blanke, C. D., et al., Phase III randomized, intergroup trial assessing imatinib mesylate at two dose levels in patients with unresectable or metastatic gastrointestinal stromal tumors expressing the kit receptor tyrosine kinase: S0033. J Clin Oncol, 2008. 26(4): p. 626-32.
14. Blay, J. Y., et al., Prospective multicentric randomized phase III study of imatinib in patients with advanced gastrointestinal stromal tumors comparing interruption versus continuation of treatment beyond 1 year: the French Sarcoma Group. J Clin Oncol, 2007. 25(9): p. 1107-13.
15. Heinrich, M. C., et al., Molecular correlates of imatinib resistance in gastrointestinal stromal tumors. J Clin Oncol, 2006. 24(29): p. 4764-74.
16. Van Glabbeke, M., et al., initial and late resistance to imatinib in advanced gastrointestinal stromal tumors are predicted by different prognostic factors: a european organisation for research and treatment of cancer-Italian sarcoma group-Austraasian gastrointestinal trials groups study. J Clin Oncol, 2005. 23(24): p. 5795-5803.
17. Blay, J. Y., et al., Consensus meeting for the management of gastrointestinal stromal tumors. Report of the GIST Consensus Conference of 20-21 Mar. 2004, under the auspices of ESMO. Ann Oncol, 2005. 16(4): p. 566-78.
18. Debiec-Rychter, M., et al., KIT mutations and dose selection for imatinib in patients with advanced gastrointestinal stromal tumours. Eur J Cancer, 2006. 42(8): p. 1093-103.
19. Van Glabbeke, M., et al., Comparison of two doses of imatinib for the treatment of unresectable or metastatic gastrointestinal stromal tumors (GIST): A meta-analyis based on 1,640 patients (pts), in J Clin Oncol ASCO annual Meeting Proceedings. 2007. p. 10004.
20. Zalcberg, J. R., et al., Outcome of patients with advanced gastro-intestinal stromal tumours crossing over to a daily imatinib dose of 800 mg after progression on 400 mg. Eur J Cancer, 2005. 41(12): p. 1751-7.
21. Soria, J. C., et al., Phase 1 dose-escalation study of oral tyrosine kinase inhibitor masitinib in advanced and/or metastatic solid cancers. Eur J Cancer, 2009. 45(13): p. 2333-41.
22. Tebib, J., et al., Masitinib in the treatment of active rheumatoid arthritis: results of a multicentre, open-label, dose-ranging, phase 2a study. Arthritis Res Ther, 2009. 11(3): p. R95.
23. Van Glabbeke, M., et al., Predicting toxicities for patients with advanced gastrointestinal stromal tumours treated with imatinib: a study of the European Organisation for Research and Treatment of Cancer, the Italian Sarcoma Group, and the Australasian Gastro-Intestinal Trials Group (EORTC-ISG-AGITG). Eur J Cancer, 2006. 42(14): p. 2277-85.
24. Choi, H., et al., Correlation of computed tomography and positron emission tomography in patients with metastatic gastrointestinal stromal tumor treated at a single institution with imatinib mesylate: proposal of new computed tomography response criteria. J Clin Oncol, 2007. 25(13): p. 1753-9.
25. Lassau, N., et al., Gastrointestinal stromal tumors treated with imatinib: monitoring response with contrast-enhanced sonography. AJR Am J Roentgenol, 2006. 187(5): p. 1267-73.
26. Prior, J. O., et al., Early prediction of response to sunitinib after imatinib failure by 18F-fluorodeoxyglucose positron emission tomography in patients with gastrointestinal stromal tumor. J Clin Oncol, 2009. 27(3): p. 439-45.
27. Van den Abbeele, A. D. and R. D. Badawi, Use of positron emission tomography in oncology and its potential role to assess response to imatinib mesylate therapy in gastrointestinal stromal tumors (GISTs). Eur J Cancer, 2002. 38 Suppl 5: p. S60-5.
28. Chami, L., et al. Quantitative functional imaging by dynamic contrast enhanced ultrasonography (DCE-US) in patients with GIST treated by tyrosine kinase inhibitor (TKI). in J Clin Oncol ASCO annual Meeting Proceedings. 2008.
29. Benjamin, R. S., et al., We should desist using RECIST, at least in GIST. J Clin Oncol, 2007. 25(13): p. 1760-4.
30. Le Cesne, A., et al., Absence of progression as assessed by RECIST predicts survival in advanced gastro-intestinal tumors (GIST) treated with imatinib mesylate: analysis of the intergroup EORTC-ISG-AGITG phase III trial. J Clin Oncol, in press.
31. Demetri, G. D., et al., Efficacy and safety of sunitinib in patients with advanced gastrointestinal stromal tumour after failure of imatinib: a randomised controlled trial. Lancet, 2006. 368(9544): p. 1329-38.
32. Siesser, P. M. and S. K. Hanks, The signaling and biological implications of FAK overexpression in cancer. Clin Cancer Res, 2006. 12(11 Pt 1): p. 3233-7.

1. A method for the treatment of a subject with Gastrointestinal Stromal Tumours (GIST), wherein said method comprises the administration of an effective amount of masitinib or a pharmaceutically acceptable salt thereof, in particular of masitinib mesylate, to said subject.

2. The method of claim 1, wherein the treatment is for treating or preventing cancer cell metastasis.

3. The method of claim 1, wherein said treatment comprises the oral administration of masitinib or a pharmaceutically acceptable salt thereof, in particular of masitinib mesylate, to a subject in need thereof.

4. The method of claim 1, wherein said effective amount of masitinib or a pharmaceutically acceptable salt thereof, in particular of masitinib mesylate, is a daily dose depending on the patient weight.

5. The method of claim 1, wherein said effective amount of masitinib or a pharmaceutically acceptable salt thereof, in particular of masitinib mesylate, is a daily dose below 18 mg/kg of subject weight.

6. The method of claim 1, wherein said effective amount of masitinib or a pharmaceutically acceptable salt thereof, in particular of masitinib mesylate, is for a first-line therapy a daily dose from 6 mg/kg to 9 mg/kg of subject weight.

7. The method of claim 1, wherein said effective amount of masitinib or a pharmaceutically acceptable salt thereof, in particular of masitinib mesylate, is for a first-line therapy a daily dose of 7.5 mg/kg of subject weight.

8. The method of claim 1, wherein said effective amount of masitinib or a pharmaceutically acceptable salt thereof, in particular of masitinib mesylate, is for a second-line therapy a daily dose from 10.5 to 15 mg/kg of subject weight.

9. The method of claim 1, wherein said effective amount of masitinib or a pharmaceutically acceptable salt thereof, in particular of masitinib mesylate, is for a second-line therapy a daily dose of 12.5 mg/kg of subject weight.

10. The method according to claim 1, wherein the daily dose is 7.5, 9 or 10.5 mg/kg of subject weight.

11. The method of claim 1, wherein said effective amount of masitinib or a pharmaceutically acceptable salt thereof, in particular of masitinib mesylate, is administered in two intakes a day.

12. A method for the treatment of a subject with a proliferative disease wherein a tyrosine kinase is affected, said subject having cells showing a mutant kit and/or mutant PDGFRA gene(s), comprising the administration of masitinib or a pharmaceutically acceptable salt thereof, in particular of masitinib mesylate.

13. The method of claim 12, wherein said kit mutation is a mutation in exon 9, and/or 11, and/or 13, and/or 17.

14. The method of claim 12, wherein said mutation is a mutation conferring resistance to a tyrosine kinase, and in particular to imatinib drug treatment.

15. A method for the long-term treatment of a subject with Gastrointestinal Stromal Tumours (GIST), wherein said method comprises the administration on a long-term of an effective amount of masitinib or a pharmaceutically acceptable salt thereof, in particular of masitinib mesylate, to said subject.

16. The method of claim 15, wherein said long-term treatment is a treatment over more than 12 months, and more preferably more than 2 years.

17. A method for the treatment of a subject with non-pre-treated, inoperable, locally advanced or metastatic GIST, wherein said method comprises the administration of an effective amount of masitinib or a pharmaceutically acceptable salt thereof, in particular of masitinib mesylate, to said subject.

18. The method of claim 1, wherein masitinib or a pharmaceutically acceptable salt thereof, in particular masitinib mesylate, inhibits the growth of imatinib-resistant cells.

19. The method of claim 1, wherein masitinib or a pharmaceutically acceptable salt thereof, in particular masitinib mesylate, is administrated in combination with another tyrosine kinase inhibitor.

20. The method of claim 1, wherein masitinib or a pharmaceutically acceptable salt thereof, in particular masitinib mesylate, is administrated in combination with imatinib.

21. The method of claim 1, wherein masitinib or a pharmaceutically acceptable salt thereof, in particular masitinib mesylate, is administrated to a patient in need thereof, and in particular to a patient, whose tumour is not treatable by surgery.

22. The method of claim 20, wherein masitinib or a pharmaceutically acceptable salt thereof, in particular masitinib mesylate, is administrated as a second-line treatment therapy to a patient, whose tumour is resistant to another tyrosine kinase inhibitor, and for example imatinib.

23. A method for the treatment of a subject having cells resistant to a treatment of a proliferative disease wherein tyrosine kinase is affected, said treatment comprises the administration of a tyrosine kinase inhibitor other than masitinib, said method comprising the steps of:
(i) Identifying in a subject a cell resistance to a treatment by a tyrosine kinase inhibitor other than masitinib of a proliferative disease wherein tyrosine kinase is affected;
(ii) Administering masitinib or a pharmaceutically acceptable salt thereof, in particular masitinib mesylate, to said subject.

24. The method of claim 1, wherein said subject is a human patient.

25. (canceled)

26. A pharmaceutical composition comprising masitinib or a pharmaceutically acceptable salt thereof, in particular masitinib mesylate, for the treatment of a subject with Gastrointestinal Stromal Tumours (GIST), and in particular for a method as defined in claim 1.

27. The pharmaceutical composition of claim 26, wherein said composition is an oral composition.

28. The pharmaceutical composition of claim 26, wherein said composition comprises a dose of at least 50 and lower than 150 mg, and preferably of 100 mg, of masitinib or a pharmaceutically acceptable salt thereof, in particular of masitinib mesylate to be administered to a subject.

29. The pharmaceutical composition of claim 26, wherein said composition comprises a dose of at least 150 and lower than 400 mg, and preferably of 200 mg, of masitinib or a pharmaceutically acceptable salt thereof, in particular of masitinib mesylate to be administered to a subject.

30. The pharmaceutical composition of claim 26, wherein said composition comprises a dose of masitinib or a pharmaceutically acceptable salt thereof, in particular of masitinib mesylate to be administered to a subject, wherein said dose is administered in two intakes a day.

31. (canceled)

32. Masitinib of claim 31, wherein said masitinib or a pharmaceutically acceptable salt thereof, in particular masitinib mesylate, is an inhibitor of kit mutants with mutation in exon 9, and/or 11, and/or 13, and/or 17.

33. (canceled)
